(19) United States
(12) Patent Application Publication
(10) Pub. No.: US 2026/0308252 A1
(43) Pub. Date: Oct. 8, 2026

(54) HIGH CONCENTRATION FORMULATIONS FOR MONOCLONAL ANTIBODIES

(71) Applicant: Icahn School of Medicine at Mount Sinai, New York, NY (US)

(72) Inventors: Mone Zaidi, New York, NY (US); Tony Yuen, New York, NY (US); Satish Rojekar, New York, NY (US)

(21) Appl. No.: 19/482,269

(22) PCT Filed: May 7, 2024
(86) PCT No.: PCT/US2024/028171

§ 371 Date: Nov. 7, 2025

Related U.S. Application Data
(60) Provisional application No. 63/500,849, filed on May 8, 2023.

(51) Int. Cl. C07K 16/26; A61K 47/02; A61K 47/18; A61K 47/20; A61K 47/26

(52) U.S. Cl. CPC C07K 16/26; A61K 47/02; A61K 47/183; A61K 47/20; A61K 47/26; C07K 2317/24

(57) ABSTRACT

Disclosed is a highly concentrated antibody formulation of, e.g., FSH-blocking humanized antibody, for example, MS-Hu6, exhibiting thermal, monomeric, and colloidal stability at a concentration as high as 100 mg/mL. The formulation includes antioxidant L-methionine and chelating agent disodium EDTA, improving the formulation's long-term colloidal and thermal stability.

### CROSS-REFERENCE TO RELATED APPLICATIONS

This application claims priority to U.S. Provisional Patent Application No. 63/500,849, filed May 8, 2023. The contents of this application are incorporated herein by reference in its entirety.

### STATEMENT REGARDING GOVERNMENT FUNDING

This invention was made with government support under grant AG073148 awarded by the National Institutes of Health. The government has certain rights in the invention.

### TECHNICAL FIELD

The present disclosure is directed to high concentration monoclonal antibody formulations for administration to a subject. More particularly, the disclosure is directed to high concentration MS-Hu6 monoclonal antibody formulations.

### BACKGROUND

Biotherapeutics have transformed the treatment of chronic inflammatory diseases and cancers¹⁻³, with monoclonal antibodies witnessing the most rapid growth because of their specificity and adaptability⁴⁻⁶. Monoclonal antibodies, particularly blocking antibodies, are generally of low affinity, and therefore must be administered at relatively high doses to elicit therapeutic responses^{6, 7}. Mostly given subcutaneously³, they are utilized at a maximally allowable volume of 1.5 mL⁵. It has therefore become critical to develop formulations of monoclonal antibodies at ultra-high concentrations^{3, 8}. These formulations also allow single-dose self-administration using auto-injection devices, reduced dosing frequency, and improved patient compliance^{3, 9}. However, ultra-high concentration monoclonal antibody formulations are associated with physical and chemical instabilities depending on the physicochemical properties of the specific monoclonal antibody¹⁰⁻¹². Notably, due to the higher concentrations, these formulations may be very viscous or gelatinous, leading to increased tissue back pressure and pain at the injection site^{6, 13} Manufacturing challenges for highly concentrated formulations also include difficulty in pumping due to high shear stress, clogging of the membrane, high back pressure, and aggregation during the manufacturing¹⁴⁻¹⁷.

Despite these obstacles, 34 out of 103 monoclonal antibody therapeutics approved by the U.S. Food and Drug Administration (FDA), that constitute almost one third of all approved biotherapeutics, have been developed with high protein concentrations (≥100 mg/mL). 76% of the approved formulations are administered by the subcutaneous route^{3, 8}. The very first ultra-high concentration monoclonal antibody formulation, Palivizumab (Synagis, 100 mg/mL), was approved in 1998¹⁸, followed by others including Alirocumab (150 mg/mL, Sanofi-Regeneron), Cosentyx (150 mg/mL, Novartis), Actemra (180 mg/mL, Genentech), and Belimumab (200 mg/mL, GlaxoSmithKline^{1, 6, 19}.

The main issue with highly concentrated formulations is protein-protein interactions^{5, 9} resulting from high viscosity and a crowded environment^{6, 20, 21}. Such interactions are reversible or irreversible, and may cause undesirable liquid-liquid separation, protein aggregation, or opalescence^{6, 22}. The effects may also be caused by a variety of transient interactions in the solution, including hydrophobic, electrostatic, dipole-dipole, hydrogen bonding (inter and intra), or van der Waals interactions²³⁻²⁵ via Fab-Fab²⁰ or, in rare instances, Fab-Fc²⁶ interactions between antibodies.

Proteins generally undergo aggregation using native monomers, denatured proteins, and pre-existing aggregates^{17, 27, 28}. Native monomers self-associate into the oligomers through transient interactions and regain their native structure—most such associations result in reversible aggregates. However, upon stressing, and over time, monomers can associate to form multimers, resulting in irreversible (insoluble) aggregates^{22, 28}. While 5-10% soluble aggregates are acceptable for biologic therapeutics, insoluble aggregates must be limited for stable formulations^{28, 29}. The US Pharmacopeia (USP) and the FDA have set guidelines to control aggregates in a final biologic formulation^{30, 31}. For example, sub-visible particles with a hydrodynamic radius≤50-100 μm are considered optimal^{22, 29}. Hence, per the FDA, the acceptable limit of sub-visible particulate matter in a container of ≤100 mL should be 6000 particles≥10 μm and 600 particles≥25 μm^{22, 28, 30}. Particle sizes≤10 μm can cause vascular occlusion.

### SUMMARY

An embodiment is directed to a highly concentrated antibody formulation of, e.g., FSH-blocking humanized antibody, MS-Hu6, exhibiting thermal, monomeric, and colloidal stability at a concentration as high as 100 mg/mL. The formulation includes antioxidant L-methionine and chelating agent disodium EDTA, improving the formulation's long-term colloidal and thermal stability. An embodiment includes a pharmaceutical formulation, including a monoclonal antibody, phosphate buffer, sucrose, sodium chloride, polysorbate 20, L-methionine and EDTA. Another embodiment includes a pharmaceutical formulation of 100 mg/ml of MS-Hu6 antibody, 20 mM phosphate buffer pH 6.2, 260 mM of sucrose, 1 mM of sodium chloride, 0.001% w/v polysorbate 20, 1 mM of L-methionine and 1 mM of EDTA.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-1C—Thermal, monomeric and colloidal stability of formulated MS-Hu6 at increasing concentrations. (A) Protein thermal shift assay confirmed thermostability of formulated MS-Hu6 between 0.2 mg/mL and 100 mg/mL. Compared with MS-Hu6 in PBS, the difference in melting temperature (ΔTₘ) of the constant (Fc) and antigen-binding fragments (Fab) of formulated MSHu6 was <−1° C. at all concentrations. (B) Size exclusion chromatography confirmed the monomeric nature of formulated MS-Hu6 between 1 and 100 mg/mL. Monomer loss was <1% in all concentrations. (C) Particle size distribution using dynamic light scattering yielded hydrodynamic radius (rh) and polydispersity index (PDI) of formulated MS-Hu6. Excellent colloidal stability (rh<10 nm, PDI<1) was found at concentrations from 1 to 100 mg/mL

FIG. 2A-2B—Thermal and colloidal stability of formulated MS-Hu6 is maintained in the presence of L-methionine and EDTA. To improve long-term storage stability, an antioxidant L-methionine and a chelating agent EDTA were added to formulated MS-Hu6. Addition of L-methionine and EDTA to formulated MS-Hu6 did not alter its melting temperatures (Tm) in the protein thermal shift assay (A) or the hydrodynamic radius (rh or Z-average rh) or polydispersity index (PDI) in dynamic light scattering (B).

FIG. 3A-3C—Evaluation of viscosity, turbidity and clarity of the formulated MS-Hu6 at different concentrations. Formulated MS-Hu6 showed a concentration-dependent linear increase in viscosity within the acceptable industry standard (<50 centipoise, cP) (A). Formulations were also evaluated for turbidity (opalescence) at 350 nm. Low absorbance (AU, absorbance unit) was noted at lower concentrations. However, there was a concentration-dependent increase in absorbance at 100 mg/mL formulated MS-Hu6 (B). Formulated MS-Hu6 evaluated for clarity at wavelengths of 350, 540 and 595 nm demonstrated that the formulation is clear and transparent even at higher concentrations (100 mg/mL) that correlated well with the turbidity data (C).

FIG. 4A-4C—Biophysical characterization of formulated MS-Hu6. Circular dichroism (CD) spectroscopy evaluated the secondary structure of formulated MS-Hu6. Analysis in the far UV region (190-240 nm) revealed that the predominant secondary structure in MS-Hu6 was regular β-sheets and unordered/random coils (A). Secondary structure was also confirmed at higher formulation concentrations (50 and 100 mg/mL) using Fourier-transform infrared (FTIR) spectroscopy. The amide I band peak at 1637 cm−1 (intra-molecular β-sheets) and the random coil (1642-1657 cm−1) did not shift, confirming maintenance of the native conformation in formulation (B). Thermostability was further confirmed using nano differential scanning calorimetry (Nano DSC). MS-Hu6 concentrations of 50 and 100 mg/ml had comparable Tms, indicating that the overall structure is conformationally and thermally stable at high concentrations (C).

FIG. 5A-5B—Evaluation of freeze-thaw stability of the formulated MS-Hu6 (100 mg/mL) at 80/25° C. and −80° C./37° C. for three cycles. (A) Samples were analyzed using protein thermal shift for thermostability. After storage at −80/25° C., ΔTm values for both Fc and Fab domains revealed no significant difference between each cycle and baseline. However, for samples stored at −80° C./37° C. (three cycles), while the Fc domain remained stable, there was a small change in Fab ΔTm (within acceptable limit of 1° C.). (B) Samples were also analyzed using dynamic light scattering (DLS) for colloidal stability. The major peak (Peak 1) hydrodynamic radius (rh) was found to be between 3 and 7 nm at both storage conditions (−80/25° C. and −80° C./37° C.). PDI—polydispersity index. The data suggest no or minimal reversible aggregation (<0.5%; acceptable limit: 5-10%).

FIG. 6A-6B—Stability evaluations of formulated MS-Hu6 at 100 mg/mL at 4° C. and 25° C. for 90 days. (A) Samples were tested for thermostability using the protein thermal shift assay. At 4° C., there was no noticeable Tm shift for the Fab domain compared to day 0. At 25° C., there is a slight left-shift in the Tms of both the Fab and Fc domains at day 15, followed by relatively good stability up to 90 days for Fab. However, after 90 days of incubation, there is a loss of thermostability for the Fc domain with a ΔTm of −1.51° C. (B) Samples were tested for monomer loss using size exclusion chromatography (SEC). After 90 days, all three batches at 4° C. retained an average monomeric area of 99.4%, while the average monomer area retained at 25° C. was 96.8%, which is still within the acceptable limit of >95%.

FIG. 7—FSH binding of formulated MS-Hu6. FSH binding to MS-Hu6 in PBS and formulated MS-Hu6 is shown by a right shift in Fab Tm, which documents higher thermostability of the FSH:Fab complex.

### DETAILED DESCRIPTION

We have developed a first-of-its-kind humanized FSH-blocking antibody, MS-Hu6, based on studies that have established direct actions of FSH in promoting bone loss, body fat accrual, and neurodegeneration. See, for example, WO2023/004036, hereby incorporated by reference in its entirety. MS-Hu6 reduces body fat, induces thermogenic adipose tissue, improves bone density, and prevents cognitive decline in mouse models³²⁻³⁹. MS-Hu6 was humanized from the parent murine antibody clone, Hf2, generated against a short 13-amino-acid-long epitope of the receptor-binding sequence of FSHβ⁴⁰. We have shown that MS-Hu6 binds to FSHβ and blocks its interaction with the FSH receptor (FSHR) with an affinity of 7.5 nM, comparable to trastuzumab (K_{D}~5 nM, Herceptin®)³⁷. We have also confirmed the stability of MS-Hu6 and its binding to FSH using a protein thermal shift assay (also known as differential scanning fluorimetry)^{37, 41}.

In an embodiment, disclosed is a formulated ultra-high concentration of MS-Hu6 at 100 mg/mL. Namely, the effect of antibody concentration on formulation stability has been fully characterized using the protein thermal shift assay, dynamic light scattering (DLS), and size exclusion chromatography. For long-term and optimum stability, antioxidant and chelating agent concentrations were screened using the protein thermal shift assay and DLS. In addition, the formulation was also examined for viscosity, turbidity and clarity; thermal stability using differential scanning calorimetry (DSC), freeze-thaw studies, and accelerated stability; and importantly, FSH binding. Structural integrity was also evaluated using FTIR and CD spectroscopy. These studies were carried out under Good Laboratory Practice (GLP) standards per Code of Federal Regulations (Tide 21, Part 58).

### Concentration of MS-Hu6 Formulations

The ultra-high concentration of formulated MS-Hu6 was selected using protein thermal shift for thermostability, size exclusion chromatography (SEC) for monomeric stability, and dynamic light scattering (DLS) for colloidal stability. Thermal shift determines a protein's unfolding (melting) temperature (Tₘ), which is the temperature at which 50% of molecules are denatured 4.

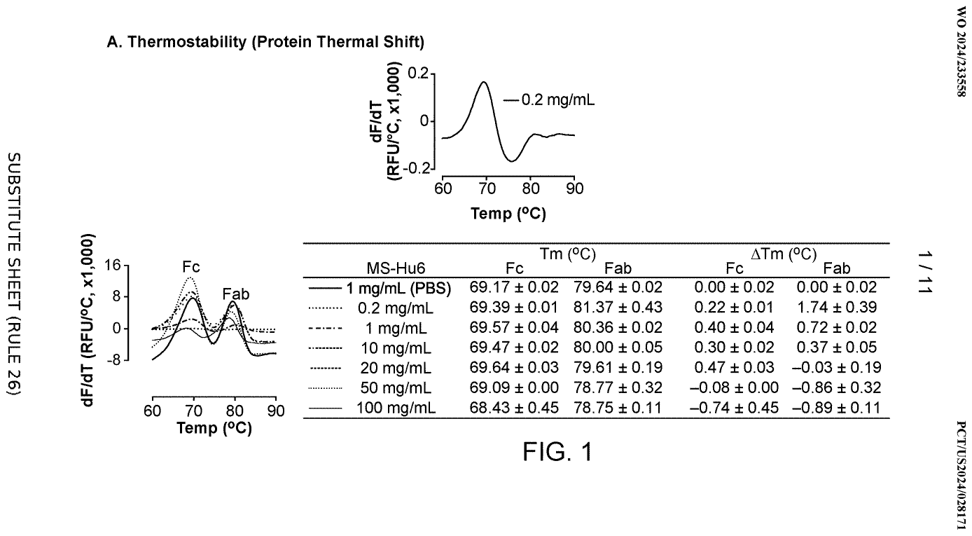

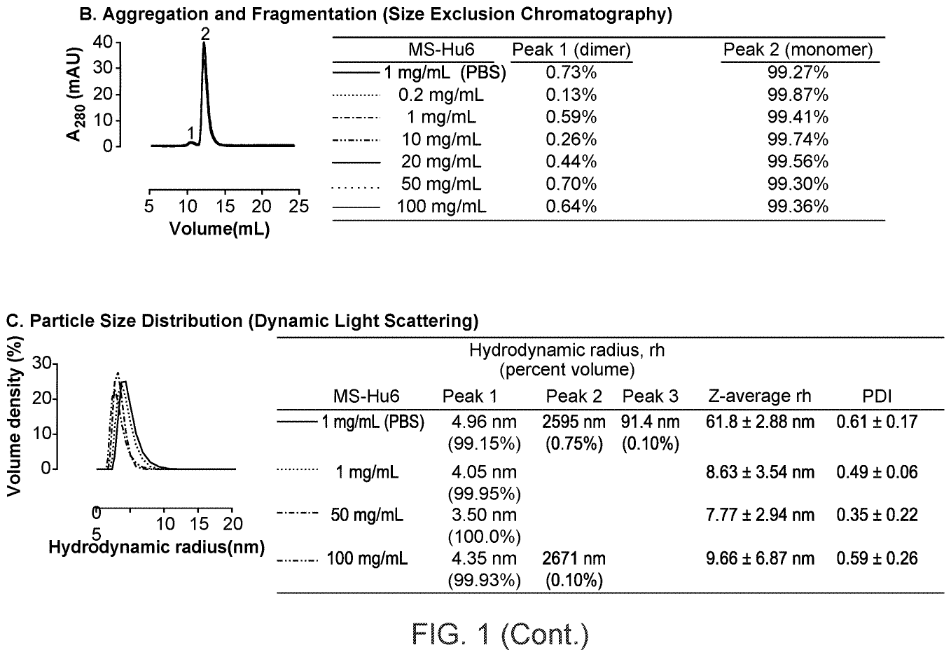

Denaturation is observed by the increase in fluorescence of SYPRO Orange, which binds to hydrophobic residues that are exposed as globular proteins unfold³⁷. Binding of ligand to the target protein results in increased stability and a higher Tₘ. Tₘ values that are higher (or more stable) indicate overall thermostability. To optimize the formulation, we increased MS-Hu6 concentration from 0.2 to 100 mg/mL (FIG. 1A). Tₘ values for the Fc and Fab domains of MS-Hu6 in PBS at 1 mg/mL were 69.17±0.02° C. and 79.64±0.02° C., respectively. Increased Tₘ values compared with MS-Hu6 in PBS were noted progressively up to 10 and 20 mg/mL for the Fab and Fc regions of formulated MS-Hu6. However, at higher concentrations (50 and 100 mg/ml), while Tₘ values dropped somewhat for the MS-Hu6 formulation compared with MS-Hu6 in PBS, the difference (or ΔTₘ) did not exceed 1° C.—which provided assurance of thermostability even at 100 mg/mL.

SEC was used to study monomeric stability of formulated MS-Hu6 based on molecular size. The percentage of monomer (peak 2) and dimer (peak 1) areas were monitored.

FIG. 1B shows that MS-Hu6 in PBS or in formulation exists mainly in its monomeric form (>99%) with a dimer area of <1%; the latter is within acceptable industry standards of <3-5%. We examined colloidal stability using DLS, a well-recognized, quick, and sensitive method to determine particle size and particle-size distribution. The method is based on light scattering due to Brownian motion. While maintaining the original structure of proteins, DLS measures the hydrodynamic diameter (rh) and polydispersity index (PDI). The samples were diluted with respective buffers (PBS or Formulation Buffer) prior to DLS. Data were collected in volume percent, with hydrodynamic radius (rh) presented for the prominent and smaller peaks (FIG. 1C). Particle size data were reported in Z-average values.

The two MS-Hu6 (in PBS) samples displayed a prominent peak (volume %~99%) with an average rh of 4.96 nm, which fell within the industry standard (rh<10 nm). However, there were two aggregated peaks with average volume percentages of <1%, but with an average rh of 91.4 and 2595 nm, respectively. The multiplicity of such peaks resulted in a high PDI, indicating varying particle size (FIG. 1C). In contrast, MS-Hu6 formulations containing 1 and 50 mg/mL MS-Hu6 displayed 99-100 volume % in a single peak, with average rh values of ~4 nm. In the 100 mg/mL formulation, however, a negligible peak (0.1% in sample 3) was associated with a large rh of 2671 nm, whereas the other samples (1 and 2) displayed single peaks with an average rh of 3.68 nm. This resulted in lower PDI values for 1 and 50 mg/L, with a similar PDI for the 100 mg/ml formulation to that of MS-Hu6 in PBS. Nonetheless, the PDI values in all cases were <1, which is the accepted cut-off. Large particles with a high rh values can form as soluble reversible aggregates, which are well accepted in biotherapeutics development at levels <10%. Thus, in all, the 100 mg/mL MS-Hu6 formulation showed an acceptable rh and PDI, suggesting that the monomeric nature of MS-Hu6 was maintained at higher concentrations.

### Selection of Antioxidant and Chelating Agents

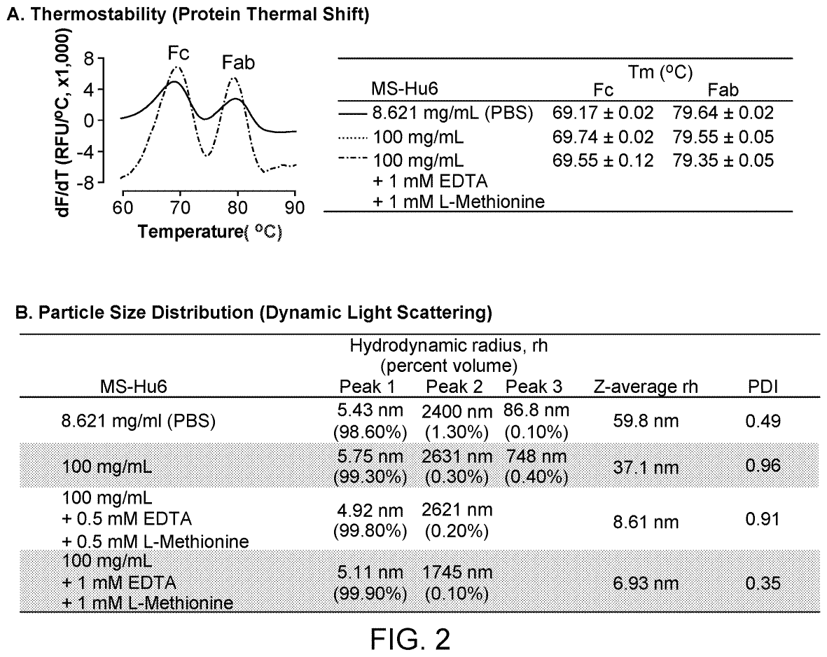

Due to the presence of trace elements and to avoid catalytic oxidation during manufacturing, disodium EDTA was tested as an excipient for formulated MS-Hu6. Furthermore, given that there are two methionine residues in the Fc domain, namely Met 33(C_{H}3) and Met 209(C_{H}3), it was determined to use L-methionine to avoid methionine oxidation during manufacturing and storage. The effect of disodium-EDTA and L-methionine on the stability of the MS-Hu6 formulation was examined using protein thermal shift and DLS. Protein thermal shift showed that compared with MS-Hu6 mg/mL) in PBS, formulated MS-Hu6 (100 mg/mL) plus 1 mM EDTA and 1 mM L-methionine showed slight increased ΔTₘ value for the Fc domain of 0.38° C. (FIG. 2 A). This suggested that methionine oxidation and catalytic oxidation were being prevented to enhance the thermostability of MS-Hu6. In contrast, there was no significant Tₘ shift in the Fab domain upon EDTA or L-methionine addition.

The effect of disodium-EDTA and L-methionine on the colloidal stability of formulated MS-hu6 was examined using DLS, with data collection in terms of % volume, rh values for the main and minor peaks, and Z-average rh values (FIG. 2 B). MS-Hu6 at 8.621 mg/mL in PBS displayed a major peak (98.6% volume) with an average rh of 5.43 nm, which fell within the industry standard (rh<10 nm). However, there were two minor aggregated peaks with <1.5% total volume, but with an average rh of 86.8 and 2400 nm, respectively. In contrast, formulated MS-Hu6 (100 mg/mL) displayed a main peak at a rh value of 5.75 nm with a higher, 99.3% volume; this size was consistent with the reported literature 4. There were two minor peaks with <0.5% volume and rh values of 748 and 2631 nm, respectively. The addition of disodium-EDTA and L-methionine to the formulation, at 0.5 mM or 1 mM each, both within the IIG limit⁴⁴⁻⁴⁶, reduced the volume of the aggregated peaks and resulted in main peaks with near-100% volume, respectively. In all cases, PDI remained <1. These data suggested that disodium-EDTA and L-methionine together improved colloidal stability, so that MS-Hu6 in the formulation existed primarily as a monomer.

### Evaluation of Viscosity, Turbidity and Clarity

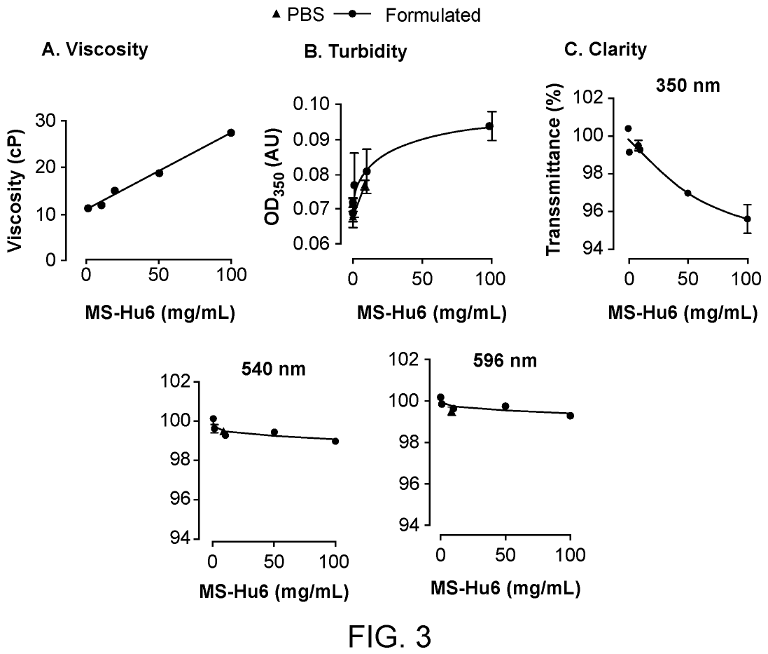

The effect of five concentrations of formulated MS-Hu6 (1, 10, 20, 50 and 100 mg/mL) was tested on dynamic viscosity, a surrogate measure for syringeability. Compared with water (3 cP) and formulation buffer (5.4 cP), formulated MS-Hu6 showed a concentration-dependent linear increase in viscosity (11.3, 12, 15, 18.8 and 27.5 cP, respectively) (FIG. 3 A). The increase in viscosity likely resulted from the crowding of MS-Hu6 molecules that is expected at high antibody concentrations 4. However, importantly, the viscosity of the formulation at all MS-Hu6 concentrations were within the acceptable industry standard (<50 cP).

Turbidity (opalescence) of various concentrations of MS-Hu6 (in formulation at 0.2, 1, 10, and 100 mg/mL) was assessed using a microplate reader at 350 nm, as well as clarity at 350, 540 and 599 nm. Comparisons were made against MS-Hu6 in PBS (8.6251 mg/mL), PBS and formulation buffer—the latter showed very low absorbance at 0.068 (±0.002), 0.072 (±0.001) and 0.077 (±0.001), respectively (FIG. 3 B). There was a concentration-dependent increase in absorbance, with a flattening at 100 mg/mL MS-Hu6 in formulation. The increase at 100 mg/mL is likely due to antibody crowding in solution and formation of reversible aggregates. However, the latter phenomenon is not consequential in biopharmaceutical product development.

Finally, there were also no differences in clarity at any wavelength except a minimal drop with formulated MS-Hu6 at 100 mg/mL (FIG. 3 C). In all, the percent transmittance for each concentration was more than >99% at the three wavelengths, suggesting that the formulations are clear between 0.2 and 50 mg/mL and slightly opalescent at 100 mg/mL. All concentrations fall into the acceptable clarity categories of Ref I and II (3-6 Nephelometric Turbidity Units)⁴⁸.

### Assessment of Structural Integrity

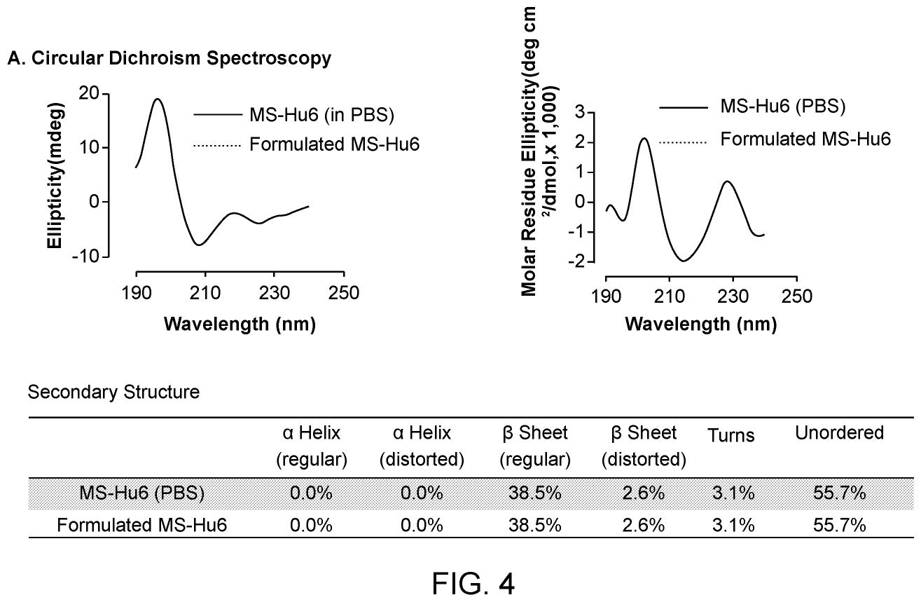

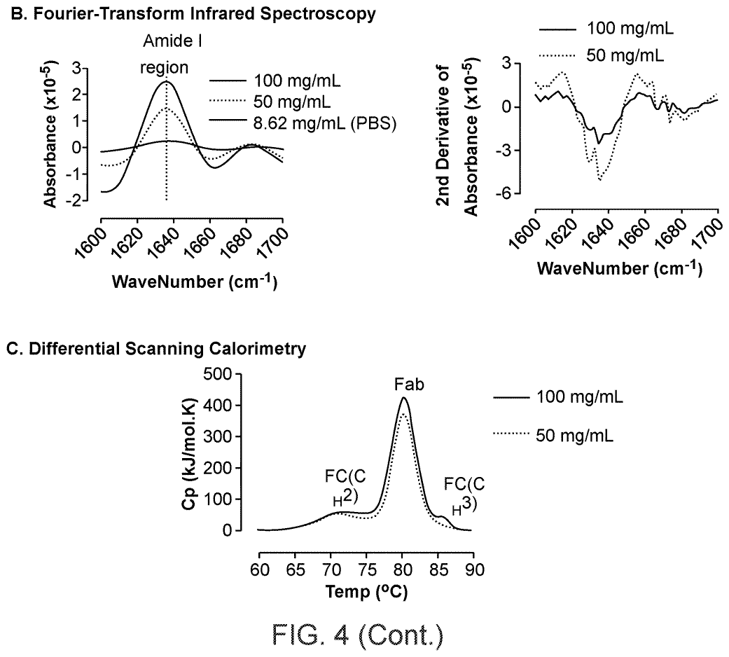

An alteration in the secondary structure or conformation of any protein indicates inactivation or denaturation. Circular Dichroism (CD) spectroscopy was used to evaluate the secondary structure of formulated MS-Hu6. As shown in FIG. 4 A and through mathematical derivations of ellipticity (millidegrees) and mean residue ellipticity, analysis in the far UV region (190-200 nm), revealed that, as with other IgG molecules, the predominant secondary structure in MS-Hu6 was regular V-sheets and unordered/random coils⁴⁹⁻⁵¹. Importantly, there was no alteration in any parameter in formulated MS-Hu6 versus MS-Hu6 in PBS (0.5 mg/mL).

However, since antibodies can be used for CD spectroscopy only at low concentrations, e.g., 0.5 mg/mL, Fourier-transform infrared (FTIR) spectroscopy was used to further study the structure of formulated MS-Hu6 at 50 and 100 mg/mL compared with MS-Hu6 in PBS. The amide I band peak at 1637 cm⁻¹, a surrogate for intra-molecular β-sheets, and the random coil structure peak at 1642-1657 cm⁻¹ did not shift, confirming maintenance of the antibody's native conformation in formulation (FIG. 4 B).

Finally, nano differential scanning calorimetry (Nano DSC) was employed to determine the protein unfolding temperatures (Tₘs). MS-Hu6 at 50 and 100 mg/ml were diluted to 5 mg/ml in formulation buffer. MS-Hu6 displayed multi-domain transitions, with the first transition representing unfolding of C_{H}2 (Fc) domain (Tₘ>71° C.), the second representing Fab unfolding (Tₘ>80.30° C.), and a third C_{H}3 (Fc) domain unfolding (Tₘ>85° C.) (FIG. 4 C). Both samples had comparable Tₘs, indicating that the overall structure is conformationally and thermally stable at high concentrations. Furthermore, the heat capacity required for the C_{H}3 unfolding was ~2-fold greater than Fab unfolding, and 7.73-8.52-fold greater than the C_{H}2 transition. This latter finding is consistent with what is observed for most IgG antibodies z.

### Rapid Freeze-Thaw Stability Evaluation

Protein instabilities, such as aggregation, are of major concern during biopharmaceutical development. Aggregation and denaturation can both take place during several phases of the product's lifecycle, including freeze-thaw, production, shipping and storage. A formal rapid freeze-thaw (F/T) study was carried out on an MS-Hu6 formulation (100 mg/mL) at −80° C./25° C. and 80° C./37° C., over three cycles, to confirm stability at extreme conditions. Samples were analyzed using protein thermal shift (PTS) and dynamic light scattering (DLS).

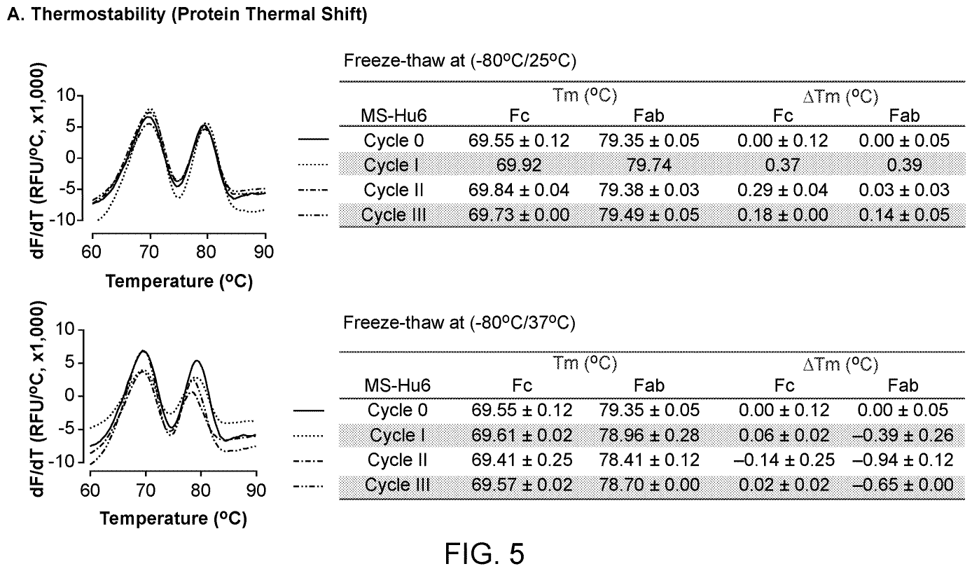

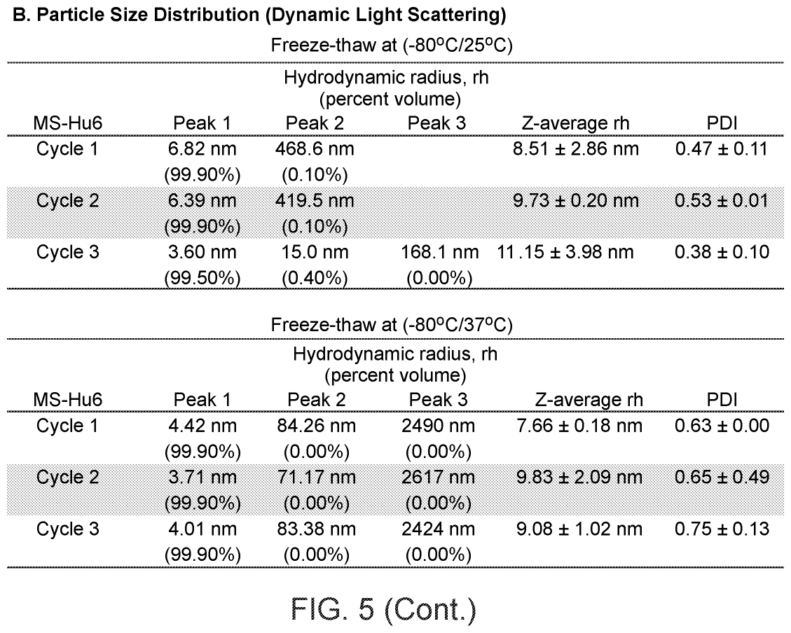

Baseline Tins for formulated MS-Hu6 (100 mg/mL) without F/T were 69.55±0.12° C. and 79.35±0.05° C. for Fc and Fab, respectively (FIG. 5 A). Samples were stored at −80° C. and thawed to 25° C. for three cycles (I, II and III). Calculations of ΔTₘ values for both Fc and Fab domains, with each F/T cycle compared with baseline revealed no significant difference between each cycle and baseline. In another experimental set, samples were stored at −80° C. and thawed to 37° C., over three cycles. While the Fc domain remained stable, there was a small, <1° C., decrease in Fab Tₘ compared with samples without F/T. The results demonstrate that formulated MS-Hu6 is stable under extreme stress and that there is minimal or no aggregation or denaturation.

All rapid F/T samples were further evaluated by DLS to determine their colloidal stability. For three cycles, the major volume peak (>99%) rh was found to be between 3 to 7 nm (FIG. 5B). The Z-average rh for both experimental sets for all cycles remained <10 nm (except cycle III of −80/25° C.), with PDIs being <1. The small increase in PDI in cycle III (−80/37° C.) is likely due to the formation of reversible aggregates resulting from Fc-fc, Fc-Fab or air-liquid interface interactions and/or reduced pH during buffer crystallization^{47, 55-57}. Nonetheless, the data are in agreement with findings for commercialized monoclonal antibody products and demonstrate no or minimal reversible aggregation (<0.4%, for −80/25° C., acceptable limit <5-10%). The formulation's excellent stability meets industry standards for product development and may improve transportability and manufacturability of MS-Hu6.

### Storage Stability Under Accelerated Conditions

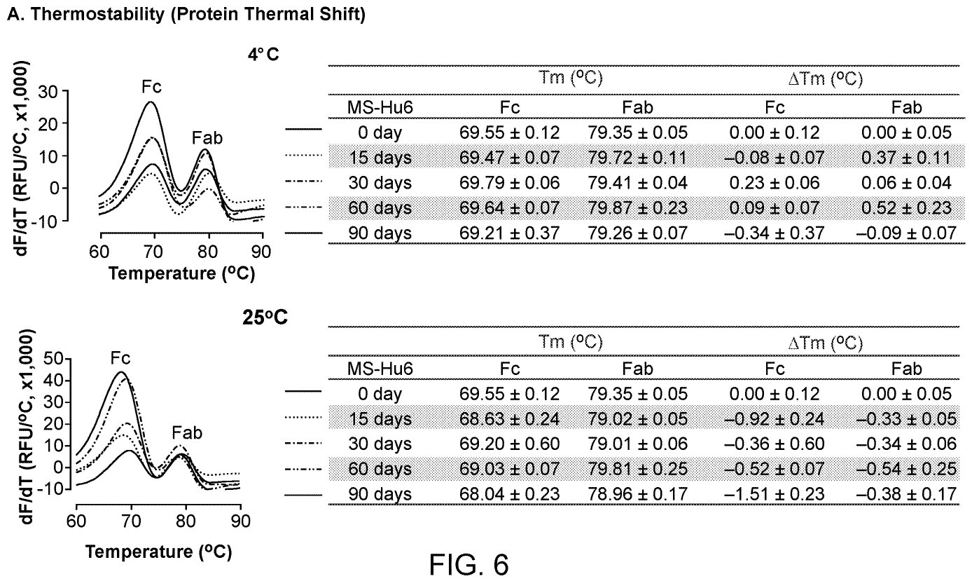

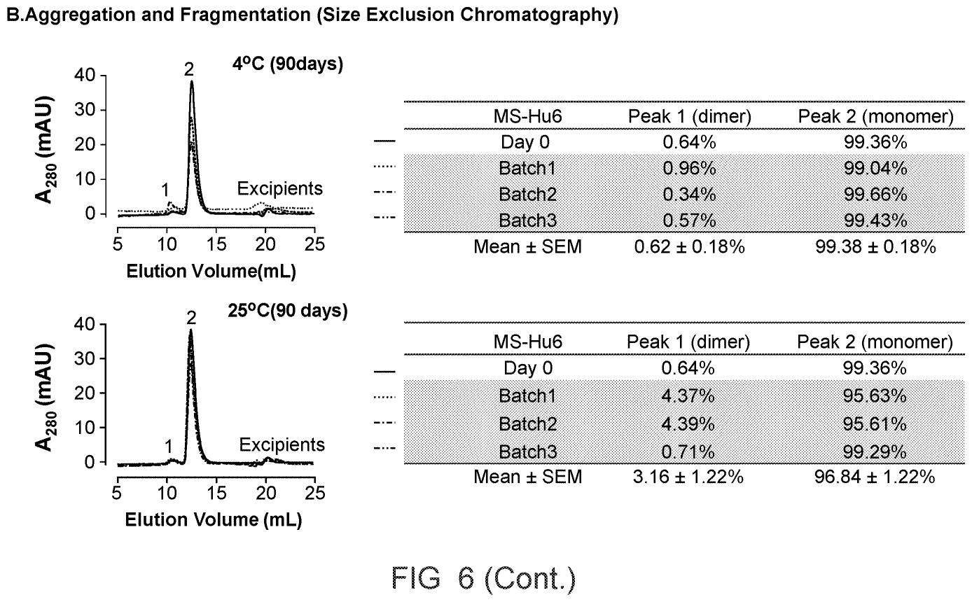

Formulated MS-Hu6 (100 mg/mL) was evaluated for storage stability at 4° C. and 25° C. for 15, 30, 60 or 90 days. Samples were tested for thermostability using protein thermal shift and for monomer loss using size exclusion chromatography (SEC). At 4° C., there was no appreciable Tₘ shift for the Fab domain compared to day 0 (FIG. 6 A). However, for the Fc domain, a very negligible left-shift of 0.34° C. was noted at 90 days, but this was within the acceptable cut-off of 1° C. When incubated at 25° C., there is a small left-shift in the Tms of both the Fab and Fc domains at day 15 (ΔTₘ 0.92° C. and 0.33° C., respectively), followed by relative good stability up to 90 days for Fab. However, during the last 90 days of incubation, there is a loss of thermostability for the Fc domain with a ΔTₘ 1.51° C. This data shows that formulated MS-Hu6 is thermostable at 4° C. (refrigerated conditions) and has excellent Fab stability in accelerated conditions at 25° C. for at least 90 days. These relatively stable Tins likely arise from the stabilizing effects of the salt, stabilizer and sugar utilized in the optimized formulation.

Monomer loss was assessed by SEC. Samples stored at 4° C. retained 99.36% of monomer area at baseline (FIG. 6 B). After 90 days at 4° C., all three batches retained an average monomeric area of 99.38±0.18%, with monomer loss<1% (acceptable limit for active structure loss<5%)^{6, 58, 59}. These findings show that MS-Hu6 in formulation (100 mg/mL) maintained monomeric stability for more than 90 days. The formation of soluble reversible aggregates, normally as a result of antibody crowding in solution or weak Fc-Fc, Fc-Fab, Fab-Fab, hydrophobic or air-liquid interactions was almost non-existent, consistent with other robust IgG formulations. Moreover, no fragmentation was observed on SEC in any samples at 4° C. for 90 days.

For samples stored at 25° C. for 90 days, the average monomer area retained was 96.84±1.22% for three batches, and monomer loss was 3.16% on average (FIG. 6 B). However, two of the samples (batches 1 and 2) lost ~4% monomer within the acceptable limit of <5%, while one sample retained 99.29% monomer. This increased monomer loss at 90 days is likely due to the formation of soluble high molecular weight species (>150 kDa). However, these species are not considered critical in biopharmaceutical development due to their reversible and transient nature.

In all, formulated MS-Hu6 at 4° C. and 25° C. for 90 days showed acceptable stability and retained maximum monomeric area in accordance with set standards.

### FSH Binding Studies

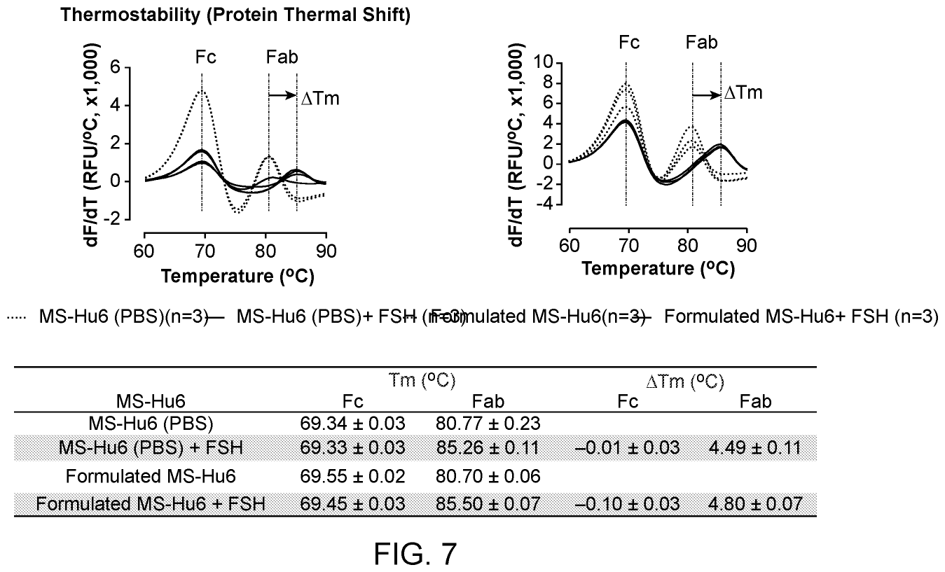

MS-Hu6 formulated according to an embodiment was evaluated for its binding to human FSH. MS-Hu6 (1 μg/μL) in formulation buffer or PBS was incubated with and without 10 μg/μL FSH, and its thermostability was measured by protein thermal shift, as before. It was expected that binding of FSH would stabilize the Fab, but not the Fc domain of MS-Hu6, and hence result in a right-shift of the Tₘ. A ΔTₘ of 4.49±0.03° C. for MS-Hu6 in PBS and 4.80±0.07° C. for formulated MS-Hu6 (FIG. 7) was noted.

There was little or no change in Tₘ in the Fc domain. These data establish conclusively the retained binding of formulated MS-Hu6 with its ligand, FSH.

The development and biophysical characterization of a formulation containing an ultra-high concentration MS-Hu6 is provided. Initially, protein thermal shift was used to find that formulated MS-Hu6 (0.2 to 100 mg/mL) had comparable Tins when compared with MS-Hu6 in PBS with ΔTₘ shifts of ≤1° C.—this confirmed thermostability for both Fc and Fab domains compatible with industry standards^{37, 41, 60}. The improved thermostability is attributed to the stabilizing effect of excipients, such as sucrose, NaCl, Tween 20, L-methionine and disodium EDTA, each of which have distinct mechanisms of action.

To assess for monomeric stability and potential aggregation, SEC was used to find that the monomeric peak was >99% at all MS-Hu6 concentrations. This suggested that formulated MS-Hu6 was monomerically stabile at higher concentrations with minimal aggregation and no fragmentation. Clinical-grade antibodies must display active protein loss of not more than 5% and secondary species<2% in a freshly reconstituted therapeutic formulations^{6, 58, 59}. SEC was complemented with DLS to ascertain aggregate formation and colloidal stability. At each MS-Hu6 concentration in formulation, the major peak volume was >99% with a rh of 3-6 nm, and peak volumes for aggregated particles of <1%; this is consistent with that reported for several clinical-grade monoclonal antibodies^{43, 61}. Of note is that soluble reversible aggregates up to 10 μm are accepted in biotherapeutics development. However, due to their potential immunogenicity or adverse reactions, particulate matter or large aggregates may need to be monitored under strict FDA guidelines^{28, 62}.

Methionine oxidation of Fc domains is a common post-translational modification that can affect antibody bioactivity and potentially induce an immunogenic response. The interface of the C_{H}2 and C_{H}3 domains of most IgG1 antibodies contains two conserved heavy chain (HC) methionine residues. The oxidation of these methionine residues reduces thermal and colloidal stability^{63,64}, and in doing so, impairs long-term storage stability (shelf-life). The antioxidant L-methionine and chelating agent, disodium-EDTA are thus used to increase the shelf-life of commercial IgG1 biotherapeutics. Free L-methionine protects methionine residues from oxidation, while EDTA increases the effectiveness of antioxidants through its ability to complex heavy metal^{44, 65}. It was determined that the addition of 1 mM L-methionine and 1 mM disodium EDTA added together resulted in thermostability in the protein thermal shift assay, and colloidal stability in DLS, with a major peak of 99.9% and rh 6.93 nm.

Viscosity is an essential parameter for any injectable dosage form, as highly viscous formulations increase back pressure and pain at injection sites. This issue is magnified for subcutaneously administered therapeutic antibodies at volumes <1.5 mL, making viscosity control a key determinant of highly concentrated formulations. It was found that an expected increase in viscosity with increasing concentrations of MS-Hu6, likely resulting from antibody crowding⁴⁷, was compounded at high concentrations, by protein-protein interactions. However, the viscosity at 100 mg/mL MS-Hu6 was considerably less than the industry maximum of 50 cPs^{66,67}. The latter is known to affect syringeability. However, viscosity can vary between 30 and 50 cPs even at high antibody concentrations (>100 mg/mL). In recent years, viscosity control has been extensively studied as a function of concentration, temperature, pH, ionic strength, and ion type⁶⁶.

Turbidity and clarity are equally important parameters in biopharmaceutical development. Increases in turbidity and reduced clarity could result from antibody overcrowding or gelation and the formation of reversible aggregates due to weak protein-protein interactions, such as Fc-Fc, Fc-Fab, hydrogen bonding, and hydrophobic interactions. It was determined that a small increase in turbidity (absorbance) of 100 mg/mL formulated MS-Hu6 at 350 nm was found. Per European Pharmacopoeia, this would be categorized as Ref II (slightly opalescent≤6 NTU) [86, 87]. In the case of clarity, percent transmittance of formulated MS-Hu6 was >99% at all wavelengths, indicative of clarity between 0.2 and 50 mg/mL, with a slight decrease at 100 mg/mL (350 nm). The clarity values fell into the categories of Ref I (3-6 NTU) and II. The majority of commercialized biopharmaceuticals have clarity values up to 12 NTU⁶⁸. Modification of secondary structure is a sign of protein inactivation or denaturation. In agreement with other IgG1 therapeutics, CD spectroscopy revealed that the predominant secondary structures in MS-Hu6 are β-sheets and unordered/random coils⁴⁹⁻⁵¹. These structures remained unaltered in the formulated version with no loss of active protein. Active protein loss in a newly reconstituted biotherapeutic formulations should be <5%, with secondary species at a maximum of 2%^{6, 58, 59}. Intact secondary structure was confirmed by FTIR, in which the amide I band peak at 1637 cm⁻¹ (representing intra-molecular V-sheets) and the random coil structure peak at 1642-1657 cm⁻¹ were unaltered in formulated MS-Hu6 (100 mg/mL) compared with MS-Hu6 in PBS. The data rule out significant inter-molecular β-sheets resulting from protein aggregation or clustering. For further confirmation, we used the nano DSC to study multidomain transitions, as a surrogate for the conformation alterations of the Fab, Fc (C_{H}2), and Fc (C_{H}3) domains. Comparison between 50 and 100 mg/mL MS-Hu6 revealed almost overlapping peaks—further confirming retained conformational stability of the respective domains.

Aggregation can occur at various stages of product development, including freeze-thaw, manufacturing, filling, shipping, and storage⁶⁹. This could destabilize and degrade the product, and thereby reduce shelf-life. A rapid freeze-thaw (F/T) study was carried out at −80° C./25° C. and −80° C./37° C. and for three cycles to confirm formulation stability at extreme conditions. ΔTₘs were found not to be significant (<1° C.) for both Fc and Fab domains in all stressed conditions, indicating that formulated MS-Hu6 was thermostable under extreme stress conditions. The data also establish the absence of aggregation. For confirmation, the samples were further evaluated by DLS for colloidal stability. All samples showed excellent colloidal stability, with minimal reversible aggregation (<0.4%, acceptable limit<5-10)⁵⁵. Moreover, the PDI or rh under stressed conditions were broadly comparable for three F/T cycles, further confirming colloidal stability.

However, a small increase in PDI was noted at the third F/T cycle at −80° C./37° C., likely attributable to reversible aggregates due to Fc-Fc, Fc-Fab, and air-liquid interface interactions^{47, 55-57}. For three cycles, these reversible aggregates remained in the 0.06 to 0.14% range, which is within acceptable limits (5-10%). This finding suggests that the ultra-high MS-Hu6 formulation retains its hydrodynamic radius at extreme storage conditions (−80° C./37° C. for three cycles).

Accelerated stability refers to the extent to which a drug retains, within specified limits, the near same properties and characteristics that it possessed at the time of manufacturing. It is a surrogate for shelf life and determines optimum storage conditions. Both Fab and Fc domains of formulated MS-Hu6 remained stable at 4° C. for 90 days (ΔTₘ<1° C. on thermal shift). Moreover, at 25° C., the Fc domain was stable for 60 days (ΔTₘ<1° C.); however, after 90 days, the Tₘ fell slightly (>1° C.), indicative of reduced stability. In contrast, the Tₘ for of the Fab domain did not change significantly (ΔTₘ−0.47° C.). SEC was used to further confirm monomeric stability after 90 days. At 4 and 25° C., all three batches retained >99% and 96.8% monomer, with loss of <1% and 3.16%, respectively (acceptable limit for active structure loss is 5%). No fragmentation was noted in any samples at 4° C. (90 days). However, monomer loss was noted after 90 days at 25° C., due to the formation of soluble dimers, with molecular weights greater than 150 kDa; the latter are not considered critical due to their reversible and transient nature. Such dimerization is induced by antibody crowding in solution, weak interactions between Fc-Fc, Fc-Fab, and Fab-Fab, and hydrophobic and air-liquid interactions^{59, 70, 71}. These soluble reversible aggregates are difficult to remove during antibody manufacturing and are therefore accepted in biotherapeutics (5-10%). However, and importantly, no fragmentation (low molecular weight species<150 kDa) was detected in any accelerated condition. Finally, MS-Hu6 in PBS and formulated MS-Hu6 were evaluated for their ability to bind FSH. Thermal shift assay revealed a right-shift for the Fab domain (ΔTₘ−4.49±0.11° C. and 4.80±0.07° C., respectively). The increased thermostability resulted from the effective binding of FSH to MS-Hu6. Expectedly, and as we have shown before, the Fc domain in either instance remained relatively unperturbed (ΔTₘ−0.1° C.)^{37,41}.

In all, we reported development of an ultra-high concentration formulation for MS-Hu6, and document its thermal, monomeric and colloidal stability, structural integrity, stability under extreme and accelerated conditions, and effective binding to the ligand, FSH. These data establish, that MS-Hu6, is developable, manufacturable, and transportable as a future therapeutic.

### Examples

### Materials and Methods

MS-Hu6 (Lot No. RO210015845) was provide by Genscript. Sodium chloride, potassium chloride and Tween 20 were purchased from Fisher Bioreagents, USA. Sodium dihydrogen phosphate, disodium hydrogen phosphate, and sucrose were from Alfa Aesar. L-Methionine and ethylenediamine-tetra-acetic acid (EDTA) (disodium salt) were procured from Sigma. Cell-culture grade water was obtained Corning. FSH (Cat #TLIH-A1110) was purchased from Golden West Biosolutions.

### Preparation of MS-Hu6 Stock

All buffers were prepared in milli-Q water (Millipore) and filtered through 0.2 μm nitrocellulose filters (Millipore). The antibody was buffer-exchanged and concentrated by centrifugation at 3000 rpm and 4° C. in a Legend X1R Centrifuge (Sorvall) using Amicon Ultra-15 centrifugal filter units with a 30 kDa molecular weight cut-off (Millipore, Cat #UFC903024). To obtain the required concentration, the antibody was diluted serially with the formulation buffer.

Concentration of MS-Hu6 before and after buffer exchange was measured by the absorbance at 280 nm using a Nanodrop ND-1000 Spectrophotometer (ThermoScientific).

### Preparing Formulated MS-Hu6 at Ultra-High Concentration

The formulation for MS-Hu6 at ultra-high concentration was prepared using several FDA-approved excipients, used within the required range per the Inactive Ingredient Limits Guidelines⁷². The final formulation contains 20 mM phosphate buffer (pH 6.2), 260 mM sucrose, 1 mM sodium chloride, 0.001% w/v Tween 20, 1 mM L-methionine and 1 mM EDTA.

### Size Exclusion Chromatography (SEC)

SEC was carried out to detect monomer loss and aggregate formation (dimer, trimer and multimer) using an AKTA Pure Fast Performance Liquid Chromatography System (Cytiva). Prepacked SEC columns (Superdex 200 10/300 GL 1×30 cm, particle diameter 13 μm) with TSKgel guard column SwXL (6 mmx 40 mm) were used. MS-Hu6 samples were diluted to 1 mg/mL using the formulation buffer, loaded onto the SEC column (500 μL), and eluted isocratically at a flow rate of 0.4 mL/min. The mobile phase is composed of sodium phosphate buffer at pH 6.2, 260 mM sucrose, 0.001% w/v Tween 20, and 1 mM NaCl at 25° C. Protein concentration was measured at 280 nm. The area under absorption curve of the chromatogram was used to determine monomeric loss. The experiment was repeated twice, and representative chromatographs are reported.

### Dynamic Light Scattering (DLS)

To provide information on colloidal stability of formulated MS-Hu6, DLS was carried out to determine the size (hydrodynamic radius, rh) and homogeneity (polydispersity Index, PDI). Briefly, 20 μL formulated MS-Hu6 was diluted into 1 mL using formulation buffer or PBS and transferred to disposable 1 mL microcuvettes (Malvern Cat. ZEN0040). Using Zetasizer Nano-ZS 90 system (Malvern), the diluted sample was analyzed for 60 seconds at 90° scattering angle at 25° C. (5 cycles). The refractive index of the medium was set at 1.33 and dynamic viscosities were measured. Data were reported as Z-average of rh and PDI. All experiments were performed in duplicate, and representative particle size distribution (PSD) graphs are reported.

### Protein Thermal Shift Assay

The protein thermal shift assay was routinely used to assess the thermostability of formulated MS-Hu6. The assay utilizes Sypro Orange (Cat #01288948, ThermoFisher), which reports hydrophobic domains that are exposed during protein unfolding. Briefly, the 20 μL reaction mixture included 2.5 μL Sypro Orange dye (10×), 0.58 μL antibody samples and 16.92 μL protein thermal shift buffer (i.e., formulation buffer). The protein samples were heated from 25° C. to 95° C. at a rate of 0.3° C./sec in a StepOnePlus real-time PCR system (Applied Biosystems), and fluorescence was captured in the ROX channel. All experiments were performed intriplicate for each concentration. Tₘ was calculated based on the inflection point of the protein thermal shift melting curve. The thermal shift (ΔTₘ) between sample A and sample B was calculated using equation 1:

$$Δ {T}_{m} = {T}_{m} A - {T}_{m} B \qquad ( 1 )$$

### Viscosity Measurement

100 to 500 μL of samples, including formulated MS-Hu6 at 1, 10, 20, 50, and 100 mg/mL, were analyzed using a Spindle S12 Viscometer (Rotavisc lo-vi, IKA) at 100 rpm and 25° C. to obtain dynamic viscosity values (Centipoise, Cp).

### Rapid Freeze-Thaw Studies

Biotherapeutics are expected to experience extreme temperature conditions while being transported or stored. Freeze-thaw cycle testing is a type of stability testing that determines the robustness of an optimized formulation. Three batches of formulated MS-Hu6 (100 μL at 100 mg/mL) were subject to repeat freeze-thaws at −80° C./25° C. or −80° C./37° C. Briefly, 100 μL formulated MS-Hu6 was incubated at −80° C. for 3 hours (TSX series freezer, ThermoFisher). Samples were thawed for 30 minutes at 25° C. in an Isotemp 250 thermostat (Fisher Scientific) or at 37° C. in a water bath (PolyScience) and analyzed by dynamic light scattering or protein thermal shift assay. The protocol was repeated to confirm antibody stability for up to three freeze-thaw cycles.

### Turbidity and Clarity

Turbidity was measured by the attenuation of incident light due to scattering⁷³. At OD350, while protein molecules have no intrinsic absorbance, aggregated particles could scatter light and thus generate opalescence. Clarity was evaluated based on percent transmitted light. Briefly, 50 μL protein samples were transferred into an uncoated flat bottom 96-well plate (Cat #12565501, Fisher Scientific) and absorbance at 350 nm (for turbidity) or transmittance at 350 nm, 540 nm and 595 nm (for clarity) was measured using a CLARIOstar microplate reader (BMG) at 25° C. after background subtraction (buffer without protein). All experiments were performed in duplicate.

### Fourier Transform Infrared Spectroscopy (FTIR)

FTIR was carried out to confirm the structural integrity of formulated MS-Hu6 at high concentration. A Nicolet iS10 FTIR Spectrometer (ThermoFisher) is equipped with an attenuated total reflectance (ATR) sampling accessory and a 45° ZnSe crystal. Briefly, 10-20 μL blank reference buffer or protein samples were loaded onto the ATR sample holder, and spectra were recorded from 500 cm⁻¹ to 4500 cm⁻¹. After subtracting the reference spectra from the sample spectra, smoothened second derivatives were generated using the Savitsky-Golay 7-point, 3^{rd} order polynomial, 2^{nd} derivative algorithm (Essential FTIR software, Operant). For appropriate peak fitting, the 2^{nd} derivative peaks were multiplied by −1. The region of structural interest, Amide I, was used to assess structural integrity.

### Far-UV Circular Dichroism (CD) Spectroscopy

Far-UV circular dichroism (CD) spectroscopy was used to assess secondary structure stability and integrity of formulated MS-Hu6. For this, 0.5 mg/mL (100 μL) of antibody samples were transferred to a cuvette. CD spectra were collected at 25° C. in a 10-mm path length with a 50-nm/min scan speed and a 0.45-sec response time using a Chirascan CD spectrometer (Applied Photophysics). A bandwidth of 2 nm was used to average five scans from 180 to 250 nm at 25° C. Built-in functions were used to subtract the reference spectra from the respective sample spectra, followed by smoothening using the Savitzky-Golay function. Equations 2 and 3 were used to compute the molar Ellipticity [Θ] and Mean Residue Ellipticity [MRE]:

$$[ θ ] = \frac{θ ( mdeg )}{10 + [ residues ] ( M ) + 1 ( cm )} \qquad ( 2 )$$

$$[ Residues ] ( M ) = [ Protein ] ( M ) * n \qquad ( 3 )$$

where Θ is the measured/observed ellipticity (in millidegrees) at a particular wavelength, l is the cuvette's path length (in cm), [protein] is protein concentration, and n is the number of amino acids in each protein under consideration. DichroWeb, created in the Wallace lab at the University of London, was used to process the secondary structure data⁴. Data analysis was conducted using Contin-LL (Provencher & Glockner Method) and reference set 7⁷⁴.

### Nano Differential Scanning Calorimetry (Nano-DSC)

In addition to protein thermal shift assays, DSC was also performed to evaluate the thermal stability of formulated MS-Hu6 at high concentrations. Briefly, samples were diluted with formulation buffer to 5 mg/mL, and 500 μL of buffer (reference) or diluted sample were loaded into 24 K gold cylindrical capillary cells in a Model-602000 Nano DSC (Waters), equilibrated for 10 minutes, and heated from 25° C. to 90° C. at a rate of 1° C./min at a constant pressure of 3 atm.

To obtain the precise unfolding temperature, the specific heat capacity (Cp) of the references were subtracted from the respective protein samples followed by data fitting using the three-transition model (NanoAnalyze Software). The respective thermograms for formulated MS-Hu6 is reported.

### Accelerated Stability

Protein thermal shift assays and size exclusion chromatography were used to examine the stability of formulated MS-Hu6 at 4° C. (long term storage) or 25° C. (accelerated storage). Three batches for each condition were evaluated. Samples were stored in 0.5 mL Eppendorf tubes and collected at 0, 15, 30 and 90 days. Protein thermal shift assays were performed to assess thermal stability. To determine monomeric stability, size exclusion chromatography was carried out on samples that have been stored for 90 days at 4° C. and 25° C. Experiments were run in triplicate and representative chromatographs are reported.

### FSH Binding Assay

To confirm binding, formulated MS-Hu6 or MS-Hu6 in PBS (1 μg/μL) was incubated with or without human FSH (10 μg/μL) at room temperature for 30 min. Protein thermal shift assays using Syro Orange were performed as above. Experiments were carried out in triplicate.

As used herein, the terms “about” and “approximately” are defined as being within plus or minus 10% of a given value or state, preferably within plus or minus 5% of said value or state. The terms “bivalent” and “bi-functional” are used interchangeably herein. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### REFERENCES

1. Lu R M, Hwang Y C, Liu I J, Lee C C, Tsai H Z, Li H J, Wu H C. Development of therapeutic antibodies for the treatment of diseases. J Biomed Sci 2020; 27(1): 2-30.
2. Passot C, Mulleman D, Bejan-Angoulvant T, Aubourg A, Willot S, Lecomte T, Picon L, Goupille P, Paintaud G, Ternant D. The underlying inflammatory chronic disease influences infliximab pharmacokinetics. Mabs-Austin 2016; 8(7): 1407-1416.
3. Wang S S, Yan Y S, Ho K. U S FDA-approved therapeutic antibodies with high-concentration formulation: summaries and perspectives. Antib Ther 2021; 4(4): 262-272. An Z Q. Monoclonal antibodies—a proven and rapidly expanding therapeutic modality for human diseases. Protein Cell 2010; 1(4): 319-330.
4. Shire S J, Shahrokh Z, Liu J. Challenges in the development of high protein concentration formulations. J Pharm Sci 2004; 93(6): 1390-1402.
5. Whitaker N, Xiong J, Pace S E, Kumar V, Middaugh C R, Joshi S B, Volkin D B. A Formulation Development Approach to Identify and Select Stable Ultra-High-Concentration Monoclonal Antibody Formulations With Reduced Viscosities. J Pharm Sci 2017; 106(11): 3230-3241.
6. El Bakri A, Nelson P N, Abu Odeh R O. The state of antibody therapy. Hum Immunol 2010; 71(12): 1243-1250.
7. Storz U. Extending the market exclusivity of therapeutic antibodies through dosage patents. Mabs-Austin 2016; 8(5): 841-847. Highly Concentrated Protein Formulations: Finding Solutions for the Next Generation of Parenteral Biologics. https://bioprocessintl.com/manufacturing/formulation/highly-concentrated-protein-formulations-351576/, 2014, Accessed 04/24/2023.
8. Bramham J E, Davies S A, Podmore A, Golovanov A P. Stability of a high-concentration monoclonal antibody solution produced by liquid-liquid phase separation. Mabs-Austin 2021; 13(1).
9. Jiskoot W, Hawe A, Menzen T, Volkin D B, Crommelin D J A. Ongoing Challenges to Develop High Concentration Monoclonal Antibody-based Formulations for Subcutaneous Administration: Quo Vadis? J Pharm Sci 2022; 111(4): 861-867.
10. Klich J H, Kasse C M, Mann J L, Huang Y Q, D'Aquino A I, Grosskopf A K, Baillet J, Fuller G G, Appel E A. Stable High-Concentration Monoclonal Antibody Formulations Enabled by an Amphiphilic Copolymer Excipient. Adv Ther—Germany 2022.
11. Jezek J, Rides M, Derham B, Moore J, Cerasoli E, Simler R, Perez-Ramirez B. Viscosity of concentrated therapeutic protein compositions. Adv Drug Deliver Rev 2011; 63(13): 1107-1117.
12. Thomas C R, Nienow A W, Dunnill P. Action of Shear on Enzymes—Studies with Alcohol—Dehydrogenase. Biotechnol Bioeng 1979; 21(12): 2263-2278.
13. Bee J S, Stevenson J L, Mehta B, Svitel J, Pollastrini J, Platz R, Freund E, Carpenter J F, Randolph T W. Response of a Concentrated Monoclonal Antibody Formulation to High Shear. Biotechnol Bioeng 2009; 103(5): 936-943.
14. Hung J J, Borwankar A U, Dear B J, Truskett T M, Johnston K P. High concentration tangential flow ultrafiltration of stable monoclonal antibody solutions with low viscosities. J Membrane Sci 2016; 508: 113-126.
15. Goswami S, Wang W, Arakawa T, Ohtake S. Developments and Challenges for mAb-Based Therapeutics. Antibodies 2013; 2(4): 452-500.
16. Geskey J M, Thomas N J, Brummel G L. Palivizumab: a review of its use in the protection of high risk infants against respiratory syncytial virus (RSV). Biologics 2007; 1(1): 33-43. Baldo B A. Safety of Biologics Therapy. Springer International Publishing Switzerland, 2016, p-610 pp.
17. Buck P M, Chaudhri A, Kumar S, Singh S K. Highly viscous antibody solutions are a consequence of network formation caused by domain-domain electrostatic complementarities: insights from coarse-grained simulations. Mol Pharm 2015; 12(1):127-139.
18. Arora J, Hickey J M, Majumdar R, Esfandiary R, Bishop S M, Samra H S, Middaugh C R, Weis D D, Volkin D B. Hydrogen exchange mass spectrometry reveals protein interfaces and distant dynamic coupling effects during the reversible self-association of an IgG1 monoclonal antibody. Mabs-Austin 2015; 7(3): 525-539.
19. Ratanji K D, Dearman R J, Kimber I, Thorpe R, Wadhwa M, Derrick J P. Subvisible Aggregates of Immunogenic Proteins Promote a Th1-Type Response. Toxicol Sci 2016:153(2): 258-270.
20. Esfandiary R, Parupudi A, Casas-Finet J, Gadre D, Sathish H. Mechanism of Reversible Self-Association of a Monoclonal Antibody: Role of Electrostatic and Hydrophobic Interactions. J Pharm Sci—Us 2015; 104(2): 577-586.
21. Connolly B D, Petry C, Yadav S, Demeule B, Ciaccio N, Moore J M R, Shire S J, Gokarn Y R. Weak Interactions Govern the Viscosity of Concentrated Antibody Solutions: High-Throughput Analysis Using the Diffusion Interaction Parameter. Biophys J 2012; 103(1):69-78.
22. Singh S N, Yadav S, Shire S J, Kalonia D S. Dipole-Dipole Interaction in Antibody Solutions: Correlation with Viscosity Behavior at High Concentration. Pharm Res-Dordr 2014; 31(9):2549-2558.
23. Arora J, Hu Y, Esfandiary R, Sathish H A, Bishop S M, Joshi S B, Middaugh C R, Volkin D B, Weis D D. Charge-mediated Fab-Fc interactions in an IgG1 antibody induce reversible self-association, cluster formation, and elevated viscosity. Mabs-Austin 2016; 8(8): 1561-1574.
24. Narhi L O, Schmit J, Bechtold-Peters K, Sharma D. Classification of protein aggregates. J Pharm Sci—Us 2012; 101(2): 493-498.
25. Pham N B, Meng W S. Protein aggregation and immunogenicity of biotherapeutics. Int J Pharm 2020; 585: 119523.
26. Wang W, Singh S K, Li N, Toler M R, King K R, Nema S. Immunogenicity of protein aggregates-Concerns and realities. Int J Pharmacent 2012; 431(1-2): 1-11. Inspection of Injectable Products for Visible Particulates Guidance for Industry. https://www.fda.gov/regulatory-information/search-fda-guidance-documents/inspection-injectable-products-visible-particulates, 2021, Accessed 04/24/2023. USP<788>. Particulate matter in Injections. https://www.uspnf.com/sites/default/files/usp_pdf/EN/USPNF/revisions/788_particulate_matter_in_injections.pdf, July 2012, Accessed 04/24/2023.
27. Gera S, Sant D, Haider S, Korkmaz F, Kuo T C, Mathew M, Perez-Pena H, Xie H, Chen H, Batista R, Ma K, Cheng Z, Hadelia E, Robinson C, Macdonald A, Miyashita S, Williams A, Jebian G, Miyashita H, Gumerova A et al. First-in-class humanized FSH blocking antibody targets bone and fat. Proc Natl Acad Sci USA 2020; 117(46): 28971-28979.
28. Zaidi M, Lizneva D, Kim S M, Sun L, Iqbal J, New M I, Rosen C J, Yuen T. FSH, Bone Mass, Body Fat, and Biological Aging. Endocrinology 2018; 159(10): 3503-3514.
29. Liu P, Ji Y T, Yuen T, Rendina-Ruedy E, DeMambro V E, Dhawan S, Abu-Amer W, Izadmehr S, Zhou B, Shin A C, Latif R, Thangeswaran P, Gupta A, Li J H, Shnayder V, Robinson S T, Yu Y E, Zhang X J, Yang F R, Lu P et al. Blocking FSH induces thermogenic adipose tissue and reduces body fat. Nature 2017; 546(7656): 107-+.
30. Sun L, Peng Y, Sharrow A C, Iqbal J, Zhang Z, Papachristou D J, Zaidi S, Zhu L L, Yaroslavskiy B B, Zhou H, Zallone A, Sairam M R, Kumar T R, Bo W, Braun J, Cardoso-Landa L, Schaffler M B, Moonga B S, Blair H C, Zaidi M. FSH directly regulates bone mass. Cell 2006; 125(2): 247-260.
31. Zhu L L, Blair H, Cao J, Yuen T, Latif R, Guo L, Tourkova I L, Li J, Davies T F, Sun L, Bian Z, Rosen C, Zallone A, New M I, Zaidi M. Blocking antibody to the beta-subunit of FSH prevents bone loss by inhibiting bone resorption and stimulating bone synthesis. Proc Natl Acad Sci USA 2012; 109(36): 14574-14579.
32. Gera S, Kuo T C, Gumerova A A, Korkmaz F, Sant D, DeMambro V, Sudha K, Padilla A, Prevot G, Munitz J, Teunissen A, van Leent M M T, Post T, Fernandes J C, Netto J, Sultana F, Shelly E, Rojekar S, Kumar P, Cullen L et al. FSH-blocking therapeutic for osteoporosis. Elife 2022; 11.
33. Zaidi M, Kim S M, Mathew M, Korkmaz F, Sultana F, Miyashita S, Gumerova A A, Frolinger T, Moldavski O, Barak O, Pallapati A, Rojekar S, Caminis J, Ginzburg Y, Ryu V, Davies T F, Lizneva D, Rosen C J, Yuen T. Bone circuitry and interorgan skeletal crosstalk. Elife 2023; 12.
34. Xiong J, Kang S S, Wang Z H, Liu X, Kuo T C, Korkmaz F, Padilla A, Miyashita S, Chan P M, Zhang Z H, Katsel P, Burgess J, Gumerova A, Ievleva K, Sant D, Yu S P, Muradova V, Frolinger T, Lizneva D, Iqbal J et al. FSH blockade improves cognition in mice with Alzheimer's disease. Nature 2022; 603(7901): 470-+.
35. Ji Y, Liu P, Yuen T, Haider S, He J, Romero R, Chen H, Bloch M, Kim S M, Lizneva D, Munshi L, Zhou C, Lu P, Iqbal J, Cheng Z, New M I, Hsueh A J, Bian Z, Rosen C J, Sun L et al. Epitope-specific monoclonal antibodies to FSHbeta increase bone mass. Proc Natl Acad Sci USA 2018; 115(9): 2192-2197.
36. Sant D, Rojekar S, Gera S, Pallapati A R, Gimenez-Roig J, Kuo T C, Padilla A, Korkmaz F, Cullen L, Chatterjee J, Shelly E, Meseck M, Miyashita S, Macdonald A, Sultana F, Barak O, Ryu V, Kim S M, Robinson C, Rosen C J el al. Optimizing a therapeutic humanized follicle-stimulating hormone-blocking antibody formulation by protein thermal shift assay. Ann NY Acad Sci 2023. Thermal Shift Assay (Differential Scanning Fluorimetry) Service. https://www.reactionbiology.com/services/biophysical-assays/thermal-shift-assay-tsa, Accessed 04/24/2023.
37. Akbas S, Sahin A, Calis S, Oncel H, Capan Y. Characterization of bevacizumab by dynamic light scattering while maintaining its native structure. Pharmazie 2018; 73(7):369-374.
38. Zhou S X, Evans B, Schoneich C, Singh S K. Biotherapeutic Formulation Factors Affecting Metal Leachables from Stainless Steel Studied by Design of Experiments. Aaps Pharmscitech 2012; 13(1): 284-294.
39. Rayaprolu B M, Strawser J J, Anyarambhatla G. Excipients in parenteral formulations: selection considerations and effective utilization with small molecules and biologics. Drug Dev Ind Pharm 2018; 44(10): 1565-1571.
40. Stadtman E R. Metal Ion-Catalyzed Oxidation of Proteins—Biochemical-Mechanism and Biological Consequences. Free Radical BioMed 1990; 9(4): 315-325.
41. Raut A S, Kalonia D S. Viscosity Analysis of Dual Variable Domain Immunoglobulin Protein Solutions: Role of Size, Electroviscous Effect and Protein-Protein Interactions. Pharm Res-Dordr 2016; 33(1): 155-166. PHARMACOPOEIA E. Clarity and degree of opalescence of liquids. EUROPEAN PHARMACOPOEIA 9.2, vol. 9.2. Council of Europe: France, July/2017.
42. Kelly S M, Jess T J, Price N C. How to study proteins by circular dichroism. Bba-Proteins Proteom 2005; 1751(2): 119-139.
43. Moreno M R, Tabitha T S, Nirmal J, Radhakrishnan K, Yee C H, Lim S, Venkatraman S, Agrawal R. Study of stability and biophysical characterization of ranibizumaband aflibercept. Eur J Pharm Biopharm 2016; 108: 156-167.
44. Vermeer A W P, Bremer MGEG, Norde W. Structural changes of IgG induced by heat treatment and by adsorption onto a hydrophobic Teflon surface studied by circular dichroism spectroscopy. Bba-Gen Subjects 1998; 1425(1): 1-12.
45. Ionescu R M, Vlasak J, Price C, Kirchmeier M. Contribution of variable domains to the stability of humanized IgG1 monoclonal antibodies. J Pharm Sci—Us 2008; 97(4): 1414-1426.
46. Ahrer K, Buchacher A, Iberer G, Jungbauer A. Thermodynamic stability and formation of aggregates of human immunoglobulin G characterised by differential scanning calorimetry and dynamic light scattering. J Biochem Biophys Methods 2006; 66(1-3): 73-86.
47. Garber E, Demarest S J. A broad range of Fab stabilities within a host of therapeutic IgGs. Biochem Bioph Res Co 2007; 355(3): 751-757.
48. Kaur H. Stability testing in monoclonal antibodies. Crit Rev Biotechnol 2021; 41(5): 692-714.
49. Le Guyader G, Vieillard V, Mouraud S, Do B, Marabelle A, Paul M. Stability of nivolumab in its original vials after opening and handing in normal saline bag for intravenous infusion. Eur J Cancer 2020; 135: 192-202.
50. Torrente-Lopez A, Hermosilla J, Salmeron-Garcia A, Cabeza J, Navas N. Comprehensive Analysis of Nivolumab, A Therapeutic Anti-Pd-1 Monoclonal Antibody: Impact of Handling and Stress. Pharmaceutics 2022; 14(4). NHS. A Standard Protocol for Deriving and Assessment of Stability https://wwwspsnhsuk/articles/standard-protocol-for-deriving-and-assessment-of-stability-part-2-aseptic-preparations-biopharmaceuticals-edition-2-2015-yellow-cover/2017.
51. Wang W, Singh S, Zeng D L, King K, Nema S. Antibody structure, instability, and formulation. J Pharm Sci—Us 2007; 96(1): 1-26.
52. He F, Hogan S, Latypov R F, Narhi L O, Razinkov V I. High Throughput Thermostability Screening of Monoclonal Antibody Formulations. J Pharm Sci—Us 2010; 99(4): 1707-1720.
53. Li S K, Liddell M R, Wen H. Effective electrophoretic mobilities and charges of anti-VEGF proteins determined by capillary zone electrophoresis. J Pharmaceut Biomed 2011; 55(3):603-607.
54. Wang W, Singh S, Zeng D L, King K, Nema S. Antibody structure, instability, and formulation. J Pharm Sci 2007; 96(1): 1-26.
55. Houde D, Peng Y C, Berkowitz S A, Engen J R. Post-translational Modifications Differentially Affect IgG1 Conformation and Receptor Binding. Mol Cell Proteomics 2010; 9(8): 1716-1728.
56. Liu D, Ren D, Huang H, Dankberg J, Rosenfeld R, Cocco M J, Li L, Brems D N, Remmele R L. Structure and stability changes of human IgG1 Fc as a consequence of methionine oxidation. Biochemistry—Us 2008; 47(18): 5088-5100.
57. Dion M Z, Leiske D, Sharma V K, de Zafra C L Z, Salisbury C M. Mitigation of Oxidation in Therapeutic Antibody Formulations: a Biochemical Efficacy and Safety Evaluation of N-Acetyl-Tryptophan and L-Methionine. Pharm Res-Dordr 2018; 35(11).
58. Tomar D S, Kumar S, Singh S K, Goswami S, Li L. Molecular basis of high viscosity in concentrated antibody solutions: Strategies for high concentration drug product development. Mabs-Austin 2016; 8(2): 216-228.
59. Zhang Z H, Liu Y. Recent progresses of understanding the viscosity of concentrated protein solutions. Curr Opin Chem Eng 2017; 16: 48-55.
60. Jonathan S. Kingsbury et.al. A single molecular descriptor to predict solution behavior of therapeutic antibodies. SCIENCE ADVANCES 5 Aug. 2020; 6(6: eabb0372): 1-11.
61. Jain K, Salamat-Miller N, Taylor K. Freeze-thaw characterization process to minimize aggregation and enable drug product manufacturing of protein based therapeutics. Sci Rep—Uk 2021; 11(1).
62. Lu C N, Liu D D, Liu H B, Motchnik P. Characterization of monoclonal antibody size variants containing extra light chains. Mabs-Austin 2013; 5(1): 102-113.
63. Plath F, Ringler P, Graff-Meyer A, Stahlberg H, Lauer M E, Rufer A C, Graewert M A, Svergun D, Gellermann G, Finkler C, Stracke J O, Koulov A, Schnaible V. Characterization of mAb dimers reveals predominant dimer forms common in therapeutic mAbs. Mabs-Austin 2016; 8(5): 928-940. Inactive Ingredient Search for Approved Drug Products. https://www.accessdata.fda.gov/scripts/cder/iig/index.cfm, Oct. 1, 2022, Accessed 04/24/2023.
64. Wang S, Wu G, Zhang X, Tian Z, Zhang N, Hu T, Dai W, Qian F. Stabilizing two IgG1 monoclonal antibodies by surfactants: Balance between aggregation prevention and structure perturbation. Eur J Pharm Biopharm 2017; 114: 263-277.
65. Miles A J, Ramalli S G, Wallace B A. DichroWeb, a website for calculating protein secondary structure from circular dichroism spectroscopic data. Protein Sci 2022; 31(1): 37-46.

What is claimed is:

1. A pharmaceutical formulation, comprising a monoclonal antibody, phosphate buffer, sucrose, sodium chloride, polysorbate 20, L-methionine and EDTA.

2. The pharmaceutical formulation of claim 1, wherein the monoclonal antibody is an anti-follicle-stimulating hormone (FSH) or antigen-binding portion thereof.

3. The pharmaceutical formulation of claim 2, wherein the monoclonal antibody is MS-Hu6.

4. The pharmaceutical formulation of claim 1, wherein the phosphate buffer is present at between 20 and 40 mM phosphate buffer at pH 6.2.

5. The pharmaceutical formulation of claim 1, wherein the sucrose is present at between 8 and 10% w/v.

6. The pharmaceutical formulation of claim 1, wherein the sodium chloride is present at between 1 and 100 mM.

7. The pharmaceutical formulation of claim 1, wherein the polysorbate 20 is present at between 0.001% w/v to 0.05% w/v.

8. The pharmaceutical formulation of claim 1, wherein the L-methionine is present at between 0.5 and 1 mM.

9. The pharmaceutical formulation of claim 1, wherein the EDTA is present at between 0.5 and 1 mM.

10. A pharmaceutical formulation comprising 100 mg/ml of MS-Hu6 antibody, 20 mM phosphate buffer pH 6.2, 260 mM of sucrose, 1 mM of sodium chloride, 0.001% w/v polysorbate 20, 1 mM of L-methionine and 1 mM of EDTA.

11. The pharmaceutical formulation of claim 1 having a pH of between 6.0 and 6.4.

12. The pharmaceutical formulation of claim 11, wherein the pH is 6.2.
